(19) United States
(12) Patent Application Publication
(10) Pub. No.: US 2014/0261403 A1
(43) Pub. Date: Sep. 18, 2014

(54) PHONATION ENABLED TRACHEAL APPARATUS

(71) Applicant: COVIDIEN LP, Boulder, CO (US)

(72) Inventors: John Paul Dunning, Kinnegad (IE); Bridget Valerie Lowry, Athlone (IE); Philip Michael Gillen, Longford (IE); Seamus Pio Maguire, Athlone (IE); Olaf James Lally, Galway (IE); Alan Finneran, Athlone (IE)

(73) Assignee: COVIDIEN LP, Boulder, CO (US)

(21) Appl. No.: 13/833,763

(22) Filed: Mar. 15, 2013

(51) Int. Cl. A61M 16/04; A61M 16/08

(52) U.S. Cl. CPC A61M 16/0468; A61M 16/0497; A61M 16/0434; A61M 16/0816
USPC 128/200.26

(57) ABSTRACT

A tracheal tube assembly includes an outer cannula configured to be positioned in a tracheal passageway and a first inner cannula configured to be disposed inside the outer cannula. The tracheal tube assembly further includes a flange member secured about the outer cannula, and a connector coupled to a proximal end of the outer cannula or of the first inner cannula. The outer cannula includes a length suitable for only minimally protruding outside of the tracheal passageway, if at all, to improve phonation.

### BACKGROUND

The present disclosure relates to a tracheal phonation techniques, and more particularly to a phonation enabled tracheal tube.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide range of applications exist for artificial ventilation, which may call for the use of tubes that are inserted into a patient. Such tubes may include endotracheal tubes, tracheostomy tubes, and so forth. In the latter case, the tubes are typically inserted into an opening or stoma formed in the neck and trachea of the patient. In both cases, the tubes may be used for artificial ventilation or for assisting patient ventilation. The stoma is typically formed either surgically, through a procedure such as a cricothyroidotomy, tracheostomy, or through a micro-surgical procedure such as percutaneous dilation. A tracheostomy tube is then typically inserted through the stoma, and used to provide for an artificial conduit into the patient's respiratory system.

Once the tracheostomy tube is in place, phonation or verbalizing becomes somewhat difficult, particularly when cuffed tracheostomy tubes are used. For example, the tracheostomy tube and/or cuff may interfere with the movement of air through the tracheal region used for phonation, thus blocking intelligible speech. It would be beneficial to provide for a tracheal apparatus that enables phonation, thus resulting in improved speech.

### SUMMARY

The present disclosure provides for novel tracheostomy tubes that improve phonation. In one embodiment an external cannula or “grommet” tube (e.g., tube with retaining member on distal end) may be used. The external cannula includes features useful for long term placement inside of the stoma, including a reduced length cannula suitable for minimizing or eliminating protruding into the patient's airway. The external “grommet” tube may be placed for long term use, and may act as a guide for placing a first inner cannula having one or more inflatable cuff's into the patient's airway. In certain embodiments, the first inner cannula may be manufactured in similar geometries to a traditional outer cannula of a tracheostomy tube, and may similarly include one or more inflatable cuffs. A second inner cannula may then be disposed inside of the first inner cannula. The second inner cannula may be manufactured in geometries similar to a traditional inner cannula, thus enabling the placement of the second inner cannula co-axially inside of the first inner cannula. Accordingly, the tracheostomy tube assembly described herein may include three cannulae placed co-axially with respect to each other. However, because of the reduced length of the grommet tube, only the first and/or the second inner cannulae may protrude into the patient's airway.

During use, a medical device, such as a ventilator, may be fluidly coupled to an end connector of the grommet tube tracheal assembly, thus enabling an artificial circuit into the patient airway suitable for providing gases (e.g., air) and medicine. In cases where the first inner cannula includes one or more cuffs, the one or more cuffs may be inflated to create a seal between the outside of the first inner cannula and the tracheal walls. In this manner, substances can only flow into the patient airway through the tracheal tube assembly, enabling a medical practitioner to maintain control over the type and amount of substances flowing into and out of the patient. The second inner cannula may be a disposable cannula. Indeed, the second inner cannula may be removed and replaced as needed, for example, to eliminate secretion build-up that may have otherwise blocked fluid flow. When phonation is desired, both the first and second inner cannulae may be removed but the grommet tube may be left in situ, as described in more detail below. Because the grommet tube may only minimally protrude into the airway region, improved phonation may be enabled, even with the grommet tube is left in situ. Accordingly, the grommet tube may be left on the trachea for extended periods of time, thus maintaining the stoma opening.

### BRIEF DESCRIPTION OF THE DRAWINGS

Advantages of the disclosed techniques may become apparent upon reading the following detailed description and upon reference to the drawings in which:

FIG. 1 is a perspective view of an exemplary tracheal tube in accordance with aspects of the present techniques;

FIG. 2 is a side view of certain functional parts of the tube shown in FIG. 1 prior to in situ assembly;

FIG. 3 is a side view of certain functional parts of the tube shown in FIG. 2 when disposed inside a trachea;

FIG. 4 is a side view of certain functional parts of the tube shown in FIG. 3, with a second inner cannula component removed;

FIG. 5 is a side view of certain functional parts of the tube shown in FIG. 3, with a first inner cannula component removed;

FIG. 6 is a side view of the an embodiment of an outer cannula component show in FIG. 5 including a retaining member;

FIG. 7 is a side view of the outer cannula component shown and a first inner cannula component show in FIG. 6 including threads;

FIG. 8 is a side view of an obturator and a telescoping tracheal tube assembly;

FIG. 9 is a side view of the telescoping tracheal tube assembly of FIG. 8 in a retracted position;

FIG. 10 is a side view of a telescoping tracheal tube assembly having multiple segments;

FIG. 11 is a perspective view of a radial snap cannula connector attached to a proximal end of an outer cannula, according to a specific example embodiment of the present disclosure;

FIG. 12 is a front view of the radial snap outer cannula connector illustrated in FIG. 11 and a mating inner cannula connector that is in an unlocked position with the radial snap and bayonet outer cannula connector; and

FIG. 13 is a perspective view of the radial snap outer cannula connector and the mating inner cannula connector, illustrated in FIG. 12, in the unlocked position.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

A tracheal tube assembly according to a preferred embodiment is illustrated in FIG. 1. The tracheal tube assembly 10 represented in the figures is a tracheostomy tube, although aspects of this disclosure could be applied to other tracheal tube structures, such as endotracheal tubes. The application to a tracheostomy tube is apt, however, insomuch as such tubes tend to be worn for longer periods of time, and thus may include a grommet tube or outer cannula 12, useful in maintaining a stoma during extended periods of wear. The outer cannula is illustrated as generally attached to a flange member 14. During use, the flange member 14 is disposed onto a patient's neck and secured through straps attached to one or more flange openings 16.

A first inner cannula 18 is illustrated extending both distally as well as proximally from the outer cannula 12. The first inner cannula 18 may be provided in different sizes, and may be introduced through an opening of a connector 20 of the outer cannula 12. Also depicted is a second inner cannula 22. In the depicted embodiment, the second inner cannula 22 is a disposable inner cannula that may be removed and replaced as desired, for example, to eliminate any secretions that may have accumulated in the second inner cannula 22.

During intubation, the tracheal tube assembly 10 is placed through an opening formed in the neck and trachea of a patient, and extending into the patient airway. The embodiment illustrated in the figures includes a sealing cuff 24, although in practice a wide range of tube designs may be used, including tubes having no cuffs or tubes having multiple cuffs around the first inner cannula 18. The second inner cannula 22 in the illustrated embodiment forms a conduit from which liquids or gases, including medications, may enter through a proximal opening 26 an exit through a distal opening 28 of the second outer cannula 22. The first inner cannula 18 has an outer dimension 30 allowing it to fit easily through an incision made in the neck and trachea of the patient. In practice, a range of such tubes 12, 18, 22 may be provided in different lengths and diameters to accommodate the different contours and sizes of patients and patient airways. Such tube families may include tubes designed for neonatal and pediatric patients as well as for adults. By way of example only, outer dimension 30 of the tube 18 may range from 4 mm to 16 mm.

In one embodiment, the outer cannula 12 enters the flange member 14 along a lower face 32 and protrudes through an upper face 34 of the flange member 14. When in use, the face 32 will generally be positioned against the neck of a patient, with the cannula 12 extending through an opening formed in the neck and trachea. A pair of side wings or flanges 36 extend laterally and serve to allow a strap (not shown) to hold the tube assembly in place on the patient. In the illustrated embodiment, the apertures 16 are formed in each side flange 36 to allow the passage of such a retaining device. In many applications, the flange member 14 may be taped or sutured in place as well.

In the depicted embodiment, the first inner cannula 18 includes an end connector 38. The end connector 38 is formed in accordance with industry standards to permit and facilitate connection to ventilating equipment (not shown). By way of example, standard outer dimensions may be provided as indicated at reference numeral 40 that allow a mating connector piece to be secured on the connector shown. By way of example, a presently contemplated standard dimension 40 accommodates a 15 mm connector, although other sizes and connector styles may be used. In use, then, air or other gas may be supplied to the patient through the tube assembly 10, flow into the patient through to the second inner cannula 22, and gases may be extracted from the patient. For example, the tube assembly 10 may be inserted into the patient's airways, and the cuff 24 may then be inflated through an inflation lumen 42. A pilot balloon 44 may then indicate that air is in the cuff 24, thus sealing the patient's airway. Once the tracheal tube is positioned and secured, a ventilator may be coupled to the end connector 38 and used to establish an artificial airway.

When improved phonation is desired, both the first and the second inner cannulae 12 and 22 may be removed, leaving the outer cannula 12 in place on the patient's trachea and neck. Because the outer cannula 12 includes a reduced length, the outer cannula 12 may only minimally protrude into the patient's airway. Accordingly, airflow through the patient's airway may be modestly affected, if all. The improved airflow may thus provide for enhanced phonation and patient comfort. Indeed, the patient may be able to speak without undue strain or painful vocalization, even when the outer cannula 12 is left in place. While in the depicted embodiment the outer cannula 12 is illustrated as a straight tube, in other embodiments, such as the embodiments described in more detail below with respect to FIG. 2, may include curved tubes.

FIG. 2 is a side view illustrating the outer cannula 12 having a curved portion 46, and the remainder of the tube assembly 10 components (e.g., first inner cannula 18, second inner cannula 22) prior to in situ assembly of the tracheal tube assembly 10 into the patient's trachea. Indeed, the illustrated components 12, 18, and 22 may be provided as a tube assembly kit, and the user or clinician may perform final assembly of the tracheal tube 10 by selecting the cannulae 12, 18, and 22 at desired sizes and shapes. The clinician may then insert the second inner cannula 22 into the first inner cannulae 18, and then insert both of the cannulae 18 and 22 into the outer cannula 12 prior to intubation. Portions of the cannulae 18 and 22 may extend through a distal opening 47 of the outer cannula 12. Thus assembled, the tracheal tube 10 may then be inserted into the patient's trachea.

In the depicted embodiment, the first inner cannula 18 includes an abutment member 48 disposed distal to the end connector 38. During insertion, the abutment member 48 may contact the connector 20 of the outer cannula 12, thus enabling a desired positioning of the first inner cannula 18 with respect to the outer cannula 12. Likewise, distal walls 50 of a connector 52 may contact the abutment member 48 during insertion of the second inner cannula 22, thus also enabling a desired positioning of the second inner cannula 22 with respect to the first inner cannula 18.

The outer cannula 12 may be selected to include certain properties, such as a preferred length l1 and an outer diameter (OD) d1. The length l1 may be selected to approximate a length lp of a tracheal passageway 45 used to insert the outer cannula 18 into the patient. The length l1 is measured from the distal opening 47 up to but not including the flange 14. In one usage case, the length l1 may be chosen to substantially approximate the length lp. In another usage case, the length l1 may be chosen to extend the outer cannula 12 slightly past the length lp. In this second usage case, a distal opening 46 of the outer cannula 12 may extend less than 5 mm, less than 4 mm, less than 3 mm, less than 2 mm, or less than 1 mm into the patient's airway and past the length lp. In yet another usage case, the length l1 may be selected to be slightly smaller than the length lp so that the outer cannula 12 does not protrude into the airway and instead remains fully inside the tracheal passageway 45. By only minimally protruding into the airway, or by remaining inside of the tracheal passageway 45, the outer tube 12 may only minimally interfere with airflow and phonation, if at all. By way of example, only the length l1 may be between 5 mm to 50 mm. In another example, a ratio of the diameter d1 to the length l1 may be between 1:0.5 to 1:5.

The diameter d1 may be selected to fit a variety of stoma sizes, including adult sizes, pediatric sizes, and neonatal sizes. By way of example only, the diameter d1 may be of between 2 mm to 20 mm. Likewise, the OD d2 may be selected so that the first inner cannula 18 may be disposed inside of the outer cannula 12, and the OD d3 may be selected so that the second inner cannula 22 may be disposed inside of the first inner cannula 18. That is, the OD d2 may be selected to be of a size equal to or smaller than an inner diameter (ID) 49 of the outer cannula 12, and the OD d3 may similarly selected to have a size equal to or smaller than an ID 51 of the first inner cannula 18.

In one embodiment, the length l2 may be provided to enable a more efficient respiratory circuit when the tracheal tube assembly 10 is disposed on the patient, while minimizing patient discomfort. Accordingly, l2 may include lengths of between 50 mm to 200 mm. The length l2 is measured as between a distal end (e.g., opening 28) of the tube 18 and the abutment member 48. Of course, other lengths may also be used. The length l3 of the second inner cannula 22 is usually chosen to correspond to the length l2. In some cases, the length l3 may be chosen to be greater than, or less than l2, depending on a desired application. The length l3 is measured as between a distal end (e.g., opening 28) of the tube 22 and proximal end the abutment member 48. In one embodiment, an interference fit or friction fit may be used to couple the cannulae 12, 18, and 22. In other embodiments described below, threads and/or snap connectors may be used to couple the cannulae 12, 18, and 22, in addition to or alternative to the use of interference fit couplings. As a way of example, the ratio of the length l1 to the length l2 or to the length l3 would be between 1:1.1 to 1:15.

Different techniques may be used to manufacture the tube assembly 10. The cannulae 12, 18, 22 and related components (e.g., flange 14, end connector 38) may be molded, overmolded, computer numerical control (CNC) machined, milled, or otherwise formed into their desired shapes. The tube assembly 10 components may be manufactured of materials such as a polyvinyl chloride (PVC), a PEBAX silicone, a polyurethane, thermoplastic elastomers, a polycarbonate plastic, a silicon, an acrylonitrile butadiene styrene (ABS), a polytetrafluoroethylene (PTFE). Once assembled, the tube assembly 10 may then be disposed inside of the trachea as shown in FIG. 3, and the patient then may be ventilated as desired.

FIG. 3 depicts an embodiment of the tracheal tube assembly 10 disposed on the trachea. More specifically, the flange 14 is disposed to contact the patient neck 54 and to further support the tracheal tube assembly 10, and the cannulae 12, 18, and 22 are shown as traversing a tracheal wall 56. As illustrated, the outer cannula 12 only minimally protrudes from the tracheal wall 56 into an airway 58. However, both of the inner cannulae 18 and 22 protrude through the opening 47 of the outer cannula 12 and are disposed towards a mid-region of the airway 58. By way of example, only the ratio of the length lp of the tracheal wall 56 to the length l1 is of between 1:0.9 to 1:3. When artificial ventilation is desired, the end connector 38 of the first inner cannula 18 may be coupled to a ventilator device, and the ventilator device may then direct fluid into and out of the airway 58 through the opening 28 of the second inner cannula 22. When a single ventilation passage is desired, the cuff 24 may be inflated to contact walls 56 and 60. Accordingly, the tracheal tube assembly 10 may become the single respiratory conduit into the trachea 54, suitable for use as a passage for gases and/or medicines. As mentioned previously, during use, secretions may build up in the second inner cannula 22. Accordingly, the second inner cannula 22 may be a disposable cannula suitable for a quick removal and subsequent replacement, as shown in FIG. 4.

FIG. 4 is a side view of the tracheal tube assembly 10 with the second inner cannula 22 removed. Because the figure includes like elements to FIG. 3, these elements are shown with like reference numbers. As discussed above, the second inner cannula 22 may provide a respiratory circuit into the patient's airway 58. As time goes by, secretion build-up, such as secretion build-up due to patient fluids, may accumulate in the second inner cannula 22. Accordingly, the second inner cannula 22 may be removed, as depicted, to be replaced by a new second inner cannula 22. In other embodiments, only the first inner cannula 18 and the outer cannula 12 may be used to provide for a conduit into the patient. Indeed, in some embodiments, the second inner cannula 22 may not be used, and only the first inner cannula 18 and the outer cannula 12 may be used for ventilation and/or delivery of medicine. Further in situations where improved phonation is desirable, both the inner cannulae 18 and 22 may be removed, as depicted in FIG. 5.

FIG. 5 is a side view of the tracheal tube assembly 10 with the second inner cannula 22 removed. Because the figure includes like elements to FIGS. 3 and 4, these elements are shown with like reference numbers. In the depicted embodiment, the first and the second inner cannulae 18 and 22 have been removed. However, the outer cannula 12 may remain in situ and continue to provide for long-term support of the stoma or opening used to insert the tracheal tube assembly 10. Because the outer cannula 12 only minimally protrudes into the airway 58, and in some embodiments may not protrude at all, airflows 62 and 64 may more easily traverse the airway 58, resulting in a substantially improved phonation. Further, the patient may experience increased comfort during vocalizations because of the absence of material disposed inside of the airway 58 that may have otherwise contacted walls 56 and or 60. In certain embodiments, as described in more detail with respect to FIG. 6 below, securement features may be added to the distal end of the outer cannula 12 to fasten the outer cannula 12 to the tracheal wall 56.

FIG. 6 is a side view of the outer cannula 12 including a retaining member 66. The retaining member 66 may be used to aid in maintaining the tracheal tube assembly 10 in place on the trachea 54. In one embodiment, the retaining member 66 may be an annular flange. The annular flange may be molded, overmolded, or otherwise attached onto the distal end of the outer cannula 12. In another embodiment, the retaining member 66 may be an inflatable cuff. Further, while the depicted embodiment shows the retaining member 66 outside the tracheal wall 56, in other cases, such as when it is desired that the outer cannula 12 does not extend outside of the tracheal wall 56, the retaining member 66 may be used inside of the passageway used to insert the outer cannula 12 into the trachea 54. In use, the retaining member 66 may aid in fastening the outer cannula 12 to the trachea 54. For example, during phonation, the wall 56 may move inwardly or outwardly with respect to the airway 58. Because the retaining member 66 may contact the sidewall 56, the contact may provide for a more secure retention of the outer cannula 12 inside of the trachea 54. Indeed, the outer cannula 12 may stay disposed in the trachea 54 for 30 days or more. In embodiments where the retaining member 66 is an inflatable cuff, the cuff may be deflated to aid in removing the outer cannula 12. Other securement features may be provided, useful in securing the outer cannula 12 to the first inner cannula 18, as described in more detail below with respect to FIG. 7.

FIG. 7 is a side view illustrating certain securement features that may enable a more secure attachment between the outer cannula 12 and the first inner cannula 18 while still providing for a quick and efficient removal and/or repositioning of the inner cannula 18 when the outer cannula 12 is disposed inside of the trachea 54. In the depicted embodiment, the outer cannula 12 includes a plurality of threads 68 disposed in interior walls 70. The first inner cannula 18 includes threads 72 complementary to the threads 68 of the outer cannula 12 and disposed on exterior walls 74. The threads 68 and 72 may be, for example, molded or overmolded onto the walls 70 and 74, respectively.

In use, when the first inner cannula 18 is inserted into the outer cannula 12, the threads 68 and 72 may engage each other. Then, depending on the thread type used (e.g., right-turn threading, left-turn threading), clockwise turns of the first inner cannula 18 may move the first inner cannula 18 inwardly further into the outer cannula 12, and counter-clockwise turns of the first inner cannula 18 may move the first inner cannula 18 outwardly from the outer cannula 12, or vice versa. In some embodiments, such as the depicted embodiments, the threads 68 and/or the threads 72 may be disposed on partially on the walls 70 and 74, respectively. In other embodiments, the threads 68 and/or the threads 72 may be disposed on the entirety of the walls 70 and 74, respectively. By providing for threaded embodiments, the first inner cannula 18 may be more securely and easily positioned inside of the outer cannula 12.

In some embodiments, such as the embodiment shown in a side view of FIG. 8, the end connector 38 may be disposed on the outer cannula 12 rather than on the first inner cannula 18. In some of these embodiments, the outer cannula 12 and the first inner cannula 18 may come pre-assembled from the factory to provide for a telescoping action suitable for inserting or for retracting the first inner cannula 18 into the airway 58. By “telescoping” into and out of the outer cannula 12, the inner cannula 18 may enable a faster and more efficient insertion and removal.

In the depicted embodiment, an obturator 76 may be inserted through the outer cannula 12 so that an obturator tip 78 contacts an abutment member 80 of the inner cannula 18 and pushes the inner cannula 18 inwardly, for example, into the airway 58. An abutment member 82 may be disposed in the inner walls 70 of the outer cannula 18, and be used as a distal stop for the abutment member 80. Accordingly, the first inner cannula 18 may be disposed at a desired position with respect to the outer cannula 12. When the outer cannula 12 is disposed in the trachea 54 and it is desired to insert the inner cannula 18 into the outer cannula 12, the clinician may grasp a handle 84 of the obturator 76 and push inwardly in a direction 85 until the abutment members 80 and 82 contact each other. In this position, an interference fit or friction fit force may securely couple the first inner cannula 18 to the outer cannula 12. The second inner cannula 22 may then be inserted into the outer cannula 12 and the first inner cannula 18 as described previously and used to provide for a fluid conduit into the patient. When it is desired to remove the first cannula 18 from the outer cannula 12, a retracting member 86 may be used, as described in more detail below with respect to FIG. 9.

FIG. 9 is a side view of the outer cannula 12 and the retracting member 86 disposed on the first inner cannula 18. The retracting member 86 may be provided as a string, as a cable, as a pull chord, or other flexible member. In use, the clinician may hold a proximal end 88 of the retracting member 86 and pull in an outwardly direction 90. Accordingly, the first cannula 18 may be removed from the outer cannula 12, for example, to improve phonation. In some embodiments, such as embodiments where an interference fit is used to couple the cannulae 12, 18, and 22, the retracting member 86 may be used even when the second inner cannula 22 is disposed inside of the first inner cannula 18. Indeed, the retracting member may be used to more quickly remove both cannulae 18 and 22 from the outer cannula 12.

FIG. 10 is a side view of an embodiment of the first inner cannula 18 having multiple telescoping segments 90 and 92. In the depicted embodiment, the retracting member 86 may also disposed on the segment 92 which is the segment most distal to the end connector 38 of the outer cannula 12. As mentioned previously, the obturator 76 (shown in FIG. 8), may be used to position the first inner cannula 18 into the outer cannula 12, for example, when the outer cannula 12 is disposed in the trachea 54. Indeed, the tip 78 of the obturator 76 may be chosen at a size suitable for fully traversing the segment 90 and contacting an abutment member 94 of the segment 92. The clinician may then push inwardly in the direction 85 until the abutment member 94 contacts an abutment member 96 disposed inside of the segment 90, thus fully extending the segments 90 and 92.

As mentioned above, the second inner cannula 22 may then be disposed inside of the first inner cannula 18 and the outer cannula 12, and the resulting tracheal tube assembly 10 may then be used to provide for ventilation into the airway 58. Also as mentioned previously with respect to FIGS. 8 and 9, the retracting member 86 may be used to retract the first inner cannula 18, for example, when the outer cannula 12 and the first inner cannula 18 are disposed in the trachea 54. Accordingly, the clinician may grasp the retracting member 86, pull outwardly in the direction 90 until the first inner cannula 18 is removed. Other securement features, such as radial snaps and bayonet connectors may be used to couple the cannulae 12, 18, and 22, as described in more detail below.

Referring now to FIG. 11, depicted is a schematic side view diagram of a radial snap connector 200 that may be attached to a proximal end of the outer cannula 12 and used to securely fasten to the end connector 38 of the first inner cannula 18. The radial snap and bayonet outer cannula connector, generally represented by the numeral 200, may comprise a snap flange 202 and retention tabs 204. The retention tabs 204 may be substantially parallel to a radial axis of the radial snap and bayonet outer cannula connector 200. The retention tabs 204 may further comprise first notches 206 in the retention tabs 204. These first notches 206 may be adapted for holding snap ears 308 (FIG. 12), e.g., bayonet style retention tabs. The snap flange 202 may further comprise second notches 208 adapted for further holding the snap ears 308 (FIGS. 12 and 13) once the snap ears 308 have engaged the snap flange 202. The second notches 208 may have slight bumps or locking protrusions 210 thereon for holding the snap ears 308 when the snap ears 308 are rotated thereover, e.g., locking indentations (not shown) on a face of the snap ears 308 slides over the locking protrusions 210. It is contemplated and within the scope of this disclosure that one or more retention tabs 204 may be used.

Referring to FIG. 12, the figure is a schematic front view diagram of the radial snap and bayonet outer cannula connector 200, illustrated in FIG. 11, and the mating first inner cannula end connector 38, that is in an unlocked position with the radial snap and bayonet outer cannula connector 200. The inner cannula connector 38 may be attached to the first inner cannula 18 and may comprise snap ears 308 and a ventilator hose coupling 312. The ventilator hose coupling 312 may be adapted for connection to a ventilator hose.

FIG. 13 is a schematic side view diagram of the radial snap and bayonet outer cannula connector 200 and the mating inner cannula connector 38, illustrated in FIG. 12, in the unlocked position. The snap ears 308 may be adapted to fit between the retention tabs 204, twist into the first notches 206 and snap over the second notches 208, thereby securing the inner cannula connector 38 to the snap flange 202 of the radial snap and bayonet outer cannula connector 200. In this way, the first inner cannula 18 may be inserted and secured into the outer cannula 12. Likewise, similar embodiments may be used to secure the second inner cannula 22 to the first inner cannula 12. Of course, other connector types may be used, such as squeeze and release connectors, where a proximal portion of the inner cannula 22 may be squeezed so as to release the inner cannula from the outer cannula 12.

What is claimed is:

1. A tracheal tube assembly comprising:
an outer cannula comprising an outer diameter d1 and a length l1 and configured to be positioned inside a tracheal passageway;
a first inner cannula configured to be disposed inside the outer cannula;
a flange member secured about the outer cannula; and
an end connector coupled to a proximal end of the first inner cannula, wherein a first ratio of the outer diameter d1 to the length l1 is between 1:0.5 to 1:5, and wherein the first inner cannula and the end connector form a contiguous conduit for exchanging fluid with a patient airway in operation.

2. The assembly of claim 1, comprising a second inner cannula configured to be disposed inside the first inner cannula, and wherein the second inner cannula and the end connector form the contiguous passageway for exchanging fluid with the patient airway in operation.

3. The assembly of claim 1, wherein the outer cannula comprises a retaining member disposed near a distal end of the outer cannula and configured to improve retention of the outer cannula inside of the tracheal passageway.

4. The assembly of claim 3, wherein retaining member comprises an annular flange or an inflatable cuff.

5. The assembly of claim 1, wherein the outer cannula comprises a connector disposed on a proximal end of the outer cannula and configured to contact an abutment member included in the first inner cannula when the first inner cannula is fully inserted into the outer cannula, and wherein the inner cannula is configured to be locked into the outer cannula.

6. The assembly of claim 1, wherein the outer cannula comprises a first plurality of threads disposed on an interior wall of the outer cannula, and wherein the first inner cannula comprises a second plurality of threads disposed onto an exterior wall of the first inner cannula, and wherein the first plurality of threads are configured to engage the second plurality of threads to couple the first inner cannula to the outer cannula.

7. The assembly of claim 1, wherein the outer cannula comprises a radial snap connector disposed on a proximal end of the outer cannula and configured to couple with the end connector of the first inner cannula.

8. The assembly of claim 7, wherein end connector comprises a bayonet style retention tab configured to couple to a notch of the radial snap connector to fasten the couple the end connector to the radial snap connector.

9. The assembly of claim 1, wherein the first inner cannula comprises a length l2 and wherein a second ratio of the length l1 to the length l2 is between 1:1.1 to 1:15.

10. The assembly of claim 2, wherein the second inner cannula comprises a length l3 and wherein the length l2 is equal to the length l3.

11. The assembly of claim 2, wherein the second inner cannula comprises a length l3 and wherein the length l2 is larger than the length l3.

12. A tracheal tube assembly comprising:
an outer cannula comprising a length l1 and configured to be positioned inside a tracheal passageway;
a first inner cannula configured to be disposed inside the outer cannula;
a second inner cannula comprising a length l2 and configured to be disposed inside the first inner cannula;
a flange member secured about the outer cannula; and
an end connector coupled to a proximal end of the outer cannula, wherein a first ratio of the length l1 to the length l2 is between 1:1.1 to 1:15, and wherein the second inner cannula and the end connector form a contiguous conduit for exchanging fluid with a patient airway in operation.

13. The assembly of claim 12, wherein the outer cannula comprises an outer diameter d1, and wherein a second ratio of the outer diameter d1 to the length l1 is between 1:0.5 to 1:5

14. The assembly of claim 12, wherein the first inner cannula comprises a first segment configured to be disposed inside the patient airway and having a flexible retracting member, wherein the first segment is configured to be retracted from the outer cannula by using the retracting member.

15. The assembly of claim 14, wherein the retracting member comprises a string or a cable.

16. The assembly of claim 14, wherein the first inner cannula comprises a second segment configured to be inserted through the first segment and into the patient airway.

17. A tracheal tube assembly comprising:
an outer cannula comprising an outer diameter d1 and a length l1 and configured to be positioned inside a tracheal passageway;
a first inner cannula configured to be disposed inside the outer cannula; and
a flange member secured about the outer cannula;
wherein an end connector is coupled to a proximal end of the outer cannula or of the first inner cannula, wherein a first ratio of the outer diameter d1 to the length l1 is between 1:0.5 to 1:5, and wherein the first inner cannula and the end connector form a contiguous conduit for exchanging gases with a patient airway in operation.

18. The assembly of claim 17, comprising a second inner cannula configured to be disposed inside the first inner cannula, and wherein the second inner cannula and the end connector form the contiguous passageway for exchanging fluid with the patient airway in operation.

19. The assembly of claim 17, wherein the outer cannula comprises a first plurality of threads disposed on a section of an interior wall of the outer cannula, and wherein the first inner cannula comprises a second plurality of threads partially disposed on a section of an exterior wall of the first inner cannula, and wherein the first plurality of threads are configured to engage the second plurality of threads to couple the first inner cannula to the outer cannula.

20. The assembly of claim 19, wherein the first plurality of threads are disposed on the entirety of the interior wall of the outer cannula.
